(19) United States
(12) Patent Application Publication
(10) Pub. No.: US 2022/0212009 A1
(43) Pub. Date: Jul. 7, 2022

(54) INTEGRATED HEALTH PLATFORM

(71) Applicants: VINCENT W. LI, HOLLISTON, MA (US); PETER GABRIEL, WILTSHIRE (GB); WILLIAM W. LI, HOLLISTON, MA (US); MEABH FLYNN, CORSHAM (GB)

(72) Inventors: VINCENT W. LI, HOLLISTON, MA (US); PETER GABRIEL, WILTSHIRE (GB); WILLIAM W. LI, HOLLISTON, MA (US); MEABH FLYNN, CORSHAM (GB)

(21) Appl. No.: 17/598,116

(22) PCT Filed: Mar. 25, 2020
(86) PCT No.: PCT/US2020/024718

§ 371 Date: Sep. 24, 2021

Related U.S. Application Data
(60) Provisional application No. 62/823,451, filed on Mar. 25, 2019.

(51) Int. Cl. A61N 1/36; A61N 5/06

(52) U.S. Cl. CPC A61N 1/36031; A61N 2005/0659; A61N 5/0613

(57) ABSTRACT

An integrated health platform operative to autonomously monitor a wide variety of health parameters and graphically display them in a manner intuitively straight-forward to non-medical users. The platform is further operative to promote interaction and sensory provocation in case of abnormal health readings. The platform is further operative to propose possible therapeutic actions and administer therapeutic treatments responsively to the detection of abnormal health parameters so as to address early stages of potentially serious medical conditions.

### BACKGROUND OF THE INVENTION

Although there has been an explosion of various devices to improve consumer health, there is complete fragmentation and significant limitations to their usefulness because devices follow no common technology vernacular; are of varied form, size, and design; are often not optimally designed for consumer behavior; do not communicate with each other; and have outputs that are diverse in scale and metric often too technical to interpret easily.

Furthermore, these devices are conventionally directed only for diagnostic or monitoring purposes and not to providing treatment or guidance with actionable information.

Additionally, conventional treatment commonly involves invasive surgery, administration of pharmaceuticals or chemotherapy responsively to an advanced medical condition, instead of addressing conditions at early stages when non-invasive techniques could have been effective.

Another shortcoming in today's medical practices is the lack of a simple way to tap into a curated reservoir of knowledge about evidence-based remedies that are readily accessible and understood by consumers to provide guidance on their health.

Finally, there is a tendency for the seemingly healthy to be complacent or in denial and thereby easily ignore parameters of health

Accordingly, there is need for a practical, consumer-level, integrated platform directed at detecting early-stage symptoms of potential medical conditions and a provision facilitating end user involvement.

### SUMMARY OF THE INVENTION

According to embodiments of the invention, an integrated health platform may include a processor; a hexagonal grid in electrical communication with the processor, the grid formed from a plurality of conductive hexagonal cells, two or more sensors, each of the sensors secured within one of the hexagonal cells directly or by using an adapter or communicating with the platform with or without direct contact, each sensor operative to detect a different health parameter; and a output device, the output device configured to graphically display each of the health parameters or configured to provide a sensory feedback which may be visible, audible, haptic, olfactory, or temperature-based.

In some embodiments, the output device may be configured to provide sensory feedback by connecting to another device such as a home appliance or a home system, for example, a light system, a heating system, a cooling system, or a wireless enabled voice assistant.

In some embodiments, the sensor may detect biomarkers in body fluids such as, for example, blood, urine, stool, saliva, sweat, or tears using detection technologies such as, for example, microfluidics, biophotonics, or immunoassay tools.

In some embodiments, the hexagonal array creates a garment that may lie in close proximity to, or touches, the body.

In some embodiments, a sensor may be embedded within another tangible object such as a doll that may be anthropomorphic and represent a person (tangible avatar) or represent any animal, plant, or object.

In some embodiments, the doll becomes identified only to a specific individual (personalized avatar) and any data collected and any response displayed only represents that specific individual.

In some embodiments, the health parameter data may be processed in a manner that tags normal and abnormal levels, either in excess or in deficit, and data from different parameters clustered and plotted within low-normal and high-normal threshold levels, and the upper and lower bound may be graphically displayed in an easy to interpret format such as a ring chart.

In some embodiments, the graphic designating normal range values may be indicated by an identifier such as a color; similarly, abnormal values may be distinguished by a different identifier.

In some embodiments, selecting the health parameter may provide detailed information such as historical data.

In some embodiments, the processor may be further operative: to identify one or more abnormal health parameters, each of the abnormal health parameters being non-compliant with an established health threshold, and to graphically designate the abnormal health parameters.

In some embodiments, the processor may be further operative to graphically display the abnormal health parameters. In some embodiments, the processor may be further operative to propose at least one therapeutic action to a user responsively to the abnormal health parameters.

In some embodiments, the processor may be further operative to graphically display a degree of non-compliance with the abnormal health parameters responsively to a user request. Some embodiments of the invention may further include a plurality of therapeutic devices, each of the devices embedded in one of the hexagonal cells.

In some embodiments, the processor may be further operative to initiate a plurality of therapeutic devices, each of the devices embedded in one of the hexagonal cells. In some embodiments, the processor may be further operative to actuate at least one of the therapeutic devices to administer a therapeutic treatment responsively to one or more abnormal health parameter.

In some embodiments, the therapeutic treatment may be selected from the group consisting of transference of energy such as by electricity, light, sound, infrared, electromagnetic pulse, or by bioactive or pharmaceutical administration. In some embodiments, the conductive grid is implemented as a conductive polymer.

In some embodiments, the therapeutic maneuver is the dissemination of information intended to provide guidance, taken from a curated database.

These and other aspects, features, and advantages will be understood with reference to the following description of certain embodiments of the invention.

### BRIEF DESCRIPTION OF THE DRAWINGS

The subject matter regarded as the invention is particularly pointed out and distinctly claimed in the concluding portion of the specification. The invention is best understood in view of the accompanying drawings in which:

FIG. 1 is a block diagram of an integrated health platform, according to an embodiment;

FIG. 2 is a hexagonal platform for integration of diagnostic and therapeutic devices, according to an embodiment;

FIG. 3 is an aggregated display of multiple parameters employed by the health platform of according to an embodiment.

FIG. 4 is a visual display configuration employed by health platform of FIG. 1 aggregating minimum and maximum normal values for many metrics, according to an embodiment;

FIG. 5 is a visual display configuration of groupings of various biometric parameters integrated by the health platform of FIG. 1, according to an embodiment:

FIG. 6 is an example of a tangible cue based on the systems inputs that promotes interactivity with the consumer and provokes a response through a sensory cue. This tangible cue, may be in the form of an avatar of the consumer or in the form of sensory cues in the environment, directed by the system.

FIG. 7 illustrates that the hexagonal units can create one flexible form that can accommodate devices of diverse shapes, sizes, and placement on the body.

FIG. 8A illustrates that hexagon unit array allows contoured placement in close proximity to organ of interest. FIG. 8B shows that hexagonal unit attachment points allow placement of different sensor devices. FIG. 8C shows that the hexagonal mesh is flexibly adoptable to the shapes or contours of parts of a human body.

FIG. 9 shows an example of an auxetic base structure of the hexagonal unit, according to an embodiment.

FIG. 10 illustrates an example of a hexagonal form, according to an embodiment.

FIG. 10A shows that a non-hexagon unit has gaps, which create inconsistent shapes in the mesh. FIG. 10B shows that hexagons fit together neatly without any gaps.

FIG. 11 shows that the hexagonal structure tenses in a consistent manner. The hexagon structure load is always equally distributed when fabric stretches. However, a non-hexagon unit member sags when stretched.

It will be appreciated that for the sake of clarity, elements shown in the figures may not be drawn to scale and reference numerals may be repeated in different figures to indicate corresponding or analogous elements.

### DETAILED DESCRIPTION OF THE PRESENT INVENTION

In the following detailed description, specific details are set forth in order to facilitate understanding of the invention; however, it should be understood by those skilled in the art that the present invention may be practiced without these specific details. Furthermore, well-known methods, procedures, and components have not been omitted to highlight the invention.

The present invention is a health platform operative to facilitate widespread early detection of potential medical conditions like disease, monitor medical conditions, and provide early administration of therapeutic measures or provide guidance of evidence-based remedies while also improving a general understanding of health among the non-medical practitioners.

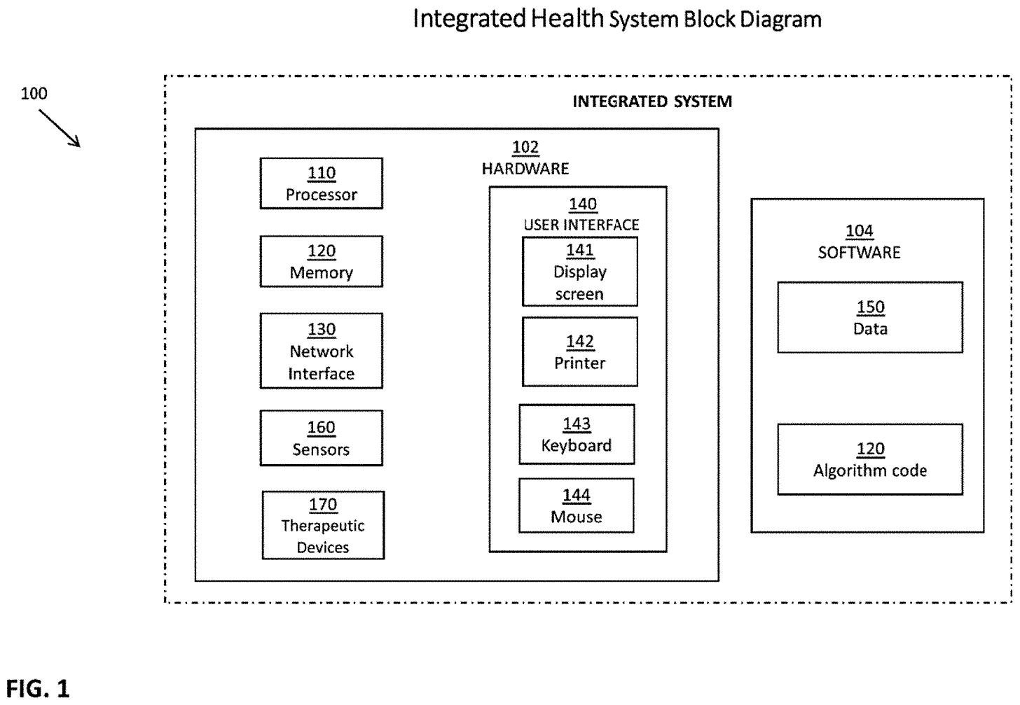

Turning now to the figures, FIG. 1 is a schematic, block diagram of an embodiment of an integrated health platform 100 including at least one processor 110 operative to execute one or more code sets, memory 120 operative to store the code sets and various data types, a network interface 130 enabling network functionality, user interface devices 140 like display screen 141, printers 142, keyboard 143, mouse 144, plus other user interface accessories, sensor array 160, and therapeutic device array 170.

As shown, system 100 includes a software module 104 including a database 105 of various types of patient data and a module of algorithm code 120 operative to process patient data. Code 120 must be executed by processor 110. Platform 100 is configured to standardize various sensor inputs and signal outputs to therapeutic devices 170.

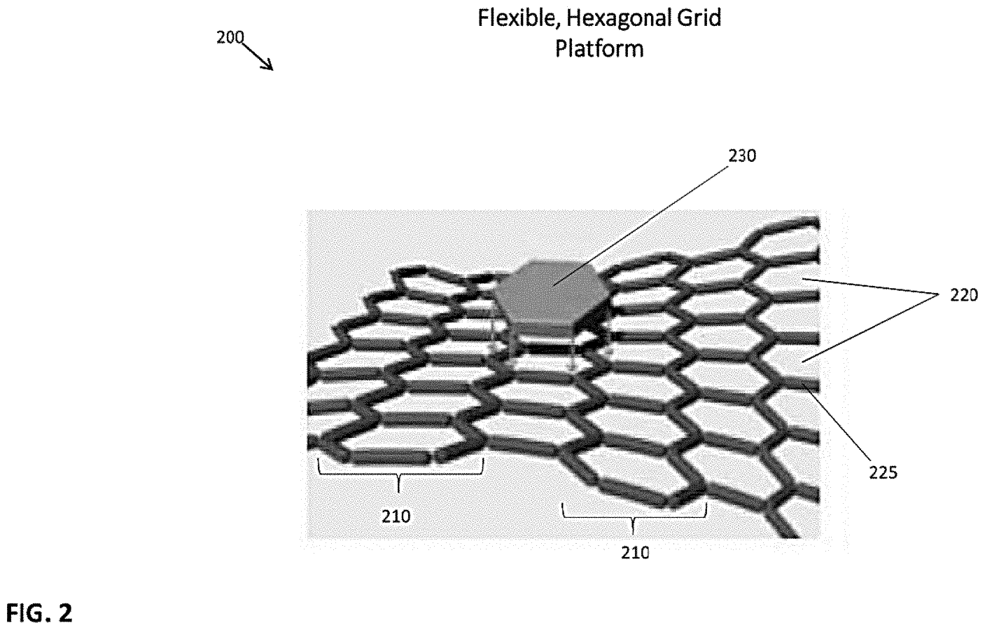

FIG. 2 is a hexagonal platform for integration of diagnostic and therapeutic devices, according to an embodiment.

As shown, a flexible hexagonal grid 200 provides an electrically conductive, scalable physical framework for securing a plurality of monitoring and therapeutic devices 230. (Device 230 is implemented as either a monitoring or a therapeutic device.)

Flexible grid 200 advantageously can be shaped to match anatomical contours while securing housing various monitoring and/or therapeutic devices. This flexibility advantageously provides the close proximity between device 230 and body surfaces required by many devices.

The hexagonal cells unit 210 allows for auxetic design in which the cells maintain their hexagonal shape under deformation thereby rendering them as the optimal shape for efficient packing, compactness, and simplicity.

Sensors and therapy devices 330 are releasably secured within grid cells 220 through a groove-ridge connection configuration embedded in device 230 and cell walls 235 in a certain embodiment, magnetic fasteners in another embodiment, or other connection configurations providing such functionality.

The hexagonal grid platform is constructed from biocompatible metals like titanium-based alloys or conductive polymers like polyacetylene, or other polymers providing biocompatibility and electrical conductivity.

The hexagonal structure provides for consistent tension among individual units with equally distributed load and reliably consistent and persistent shapes, appropriate for the integration of sensor devices.

The electrical conductivity of grid 200 facilitates cross-communication between devices at different frequencies such that a first device is responsive to a first frequency of protocol and a second device is responsive to a second frequency or protocol.

In a certain embodiment, grid devices communicate with each other directly whereas certain other embodiment devices communicate to each other through a central processor, or in another embodiment the combination of both.

Various sensors employed by the system include inter alia, sensors directed to visual heath and acuity, cerebral activity, kidney function, cardiac function, dental health breathing capacity, and blood condition.

Therapeutic device 230 is operative to confer an energy in a therapeutic form, such as light, sound, or electrical stimulation.

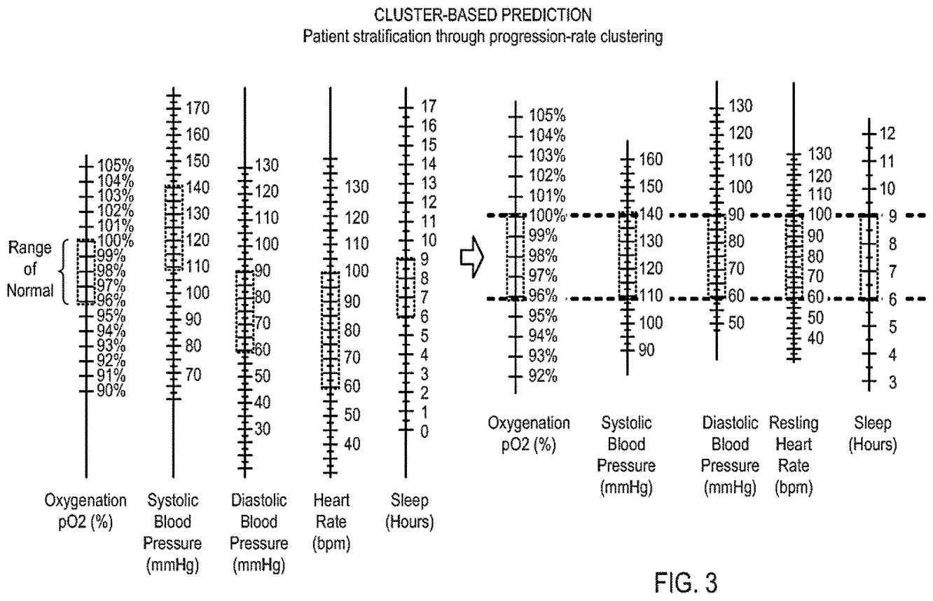

FIG. 3 is an aggregated display of multiple parameters employed by health system 100, according to an embodiment.

Key features of this readout system are directed to aggregating and integrating multiple health parameters in a manner intuitively understandable to the non-medical practitioner. System 100 is operative to display aggregate multiple health parameters so as to offer a synoptic view of health to seamlessly integrate data feeds from a multitude of biometric devices providing 50-100 separate health metrics including, for example, cardiovascular fitness, GI health, diabetes, eye health, sleep, emotion, infection, neuro-cognitive, and skin health among many others.

Furthermore, system 100 also may include metrics of external hazards to health such as EMF radiation, UV light, stress, air quality, alcohol, and heavy metals.

Health parameters are measured on scales and in units that are distinct. In order to effectively integrate different measurements, system 100 is operative to providing visualization solutions by normalizing the scales, based on the accepted range of normal minimum and maximum values, thereby, enabling graphical simplification of complex data so the minimum and maximum for each metric can be aligned.

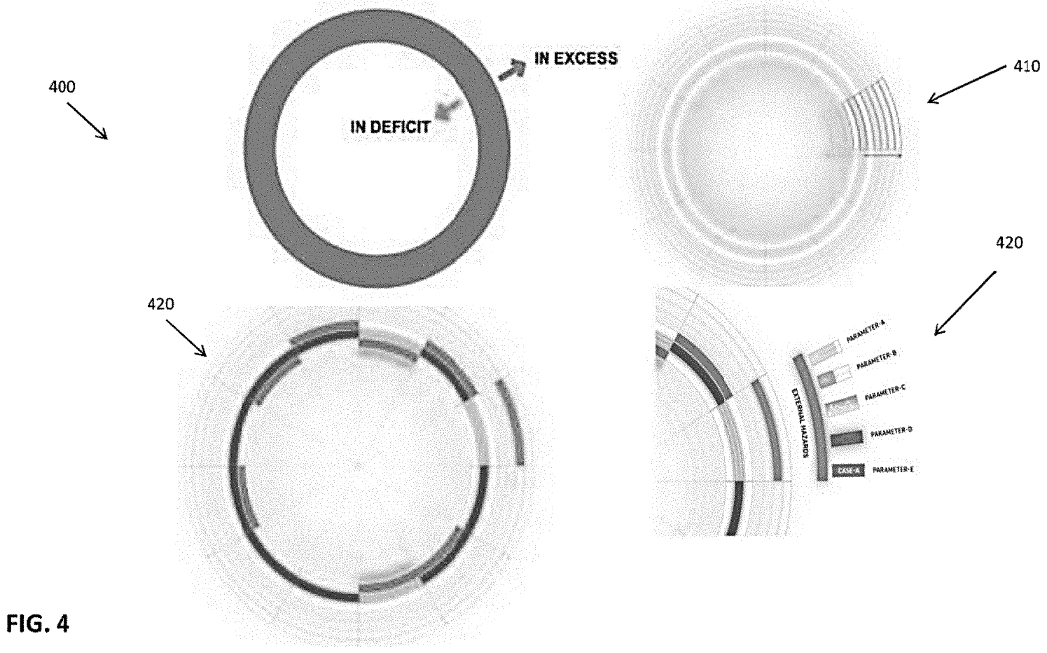

FIG. 4 is a visual display configuration employed by health platform 100 aggregating minimum and maximum normal values for many metrics creates a graphical representation that, connected at both ends, forms a ring that is simple to visualize and intuitive in interpretation. This “health prosperity ring” advantageously enables users to confirm their health biometric status through a quick glance as opposed to typical displays that challenge users with complex data. Here, a green ring becomes an easy read out conferring an “autopilot” sensibility to users to effectively remove some of the control from the hands of expert providers and transfer it to consumers.

Ring 410 also allows for scalability is that ring can be divisible into an infinite number of radians, each of which represents a health biometric. For values that are in excess of the maximum normal window, the signal of the metric would appear red, outside the perimeter of the green ring.

Ring 420 conveys the intensity of the deviation from the normal. The farther the signal is from the central green normal circle; the higher is the priority of action that may be required. Deficit of the minimum normal window, the signal would appear yellow, within the inner perimeter of the green ring.

Ring 430 depicts additional display functionality in which an abnormal health signal can be viewed, with additional detail displayed graphically.

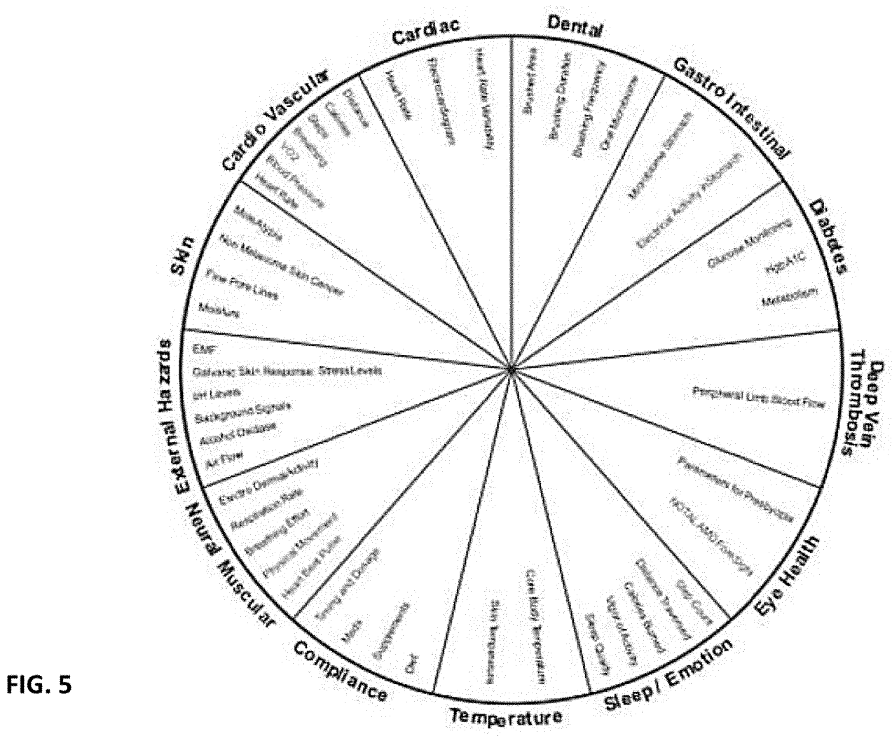

FIG. 5 is a visual display configuration of groupings of various biometric parameters integrated by health platform 100.

Health platform advantageously enables users to run on auto-pilot health scan so that it can signal when something is out of balance. Detecting an abnormal signal would allow for early diagnostics and prevention at the earliest possible time and serve as a rapid response regulator, whereby a user can assess digital data to detect and intercept disease before it becomes a serious problem. Health system 100 is configured to recommended action steps that users can take, such as suggestions of food, medicine, or appropriate lifestyle changes, can also be a powerful intervention tool and is linked to external databases such as the curated evidence-based knowledge found in the Angiogenesis Foundation's Universal Health™ Atlas or Do It Yourself Health™ Revolution initiative, for example, and stream the information to users through a mobile application so as to provide people with the opportunity to advocate for themselves and achieve better health outcomes. Below are two sample recommendations:
1) Sleep 7 hours per night to achieve 53% lower risk of adenomas of the colon. (graphical image)
2) Eat the equivalent of 1 small tomato per day to achieve a 38% lower risk of breast cancer. (graphical image)

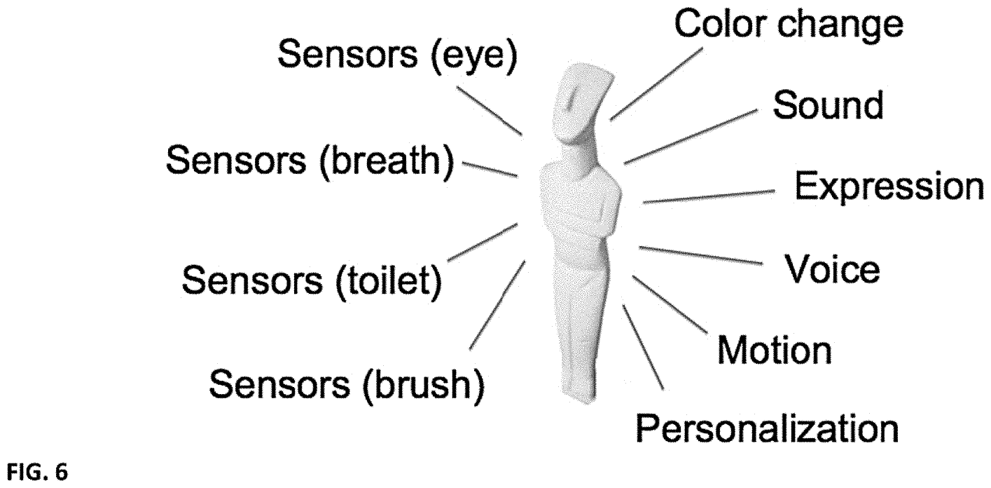

FIG. 6. is an example of a tangible anthropomorphic doll (tangible avatar) in which sensors may be embedded within the doll to capture biometric data and the object itself is able to display a number of sensory responses based on the data.

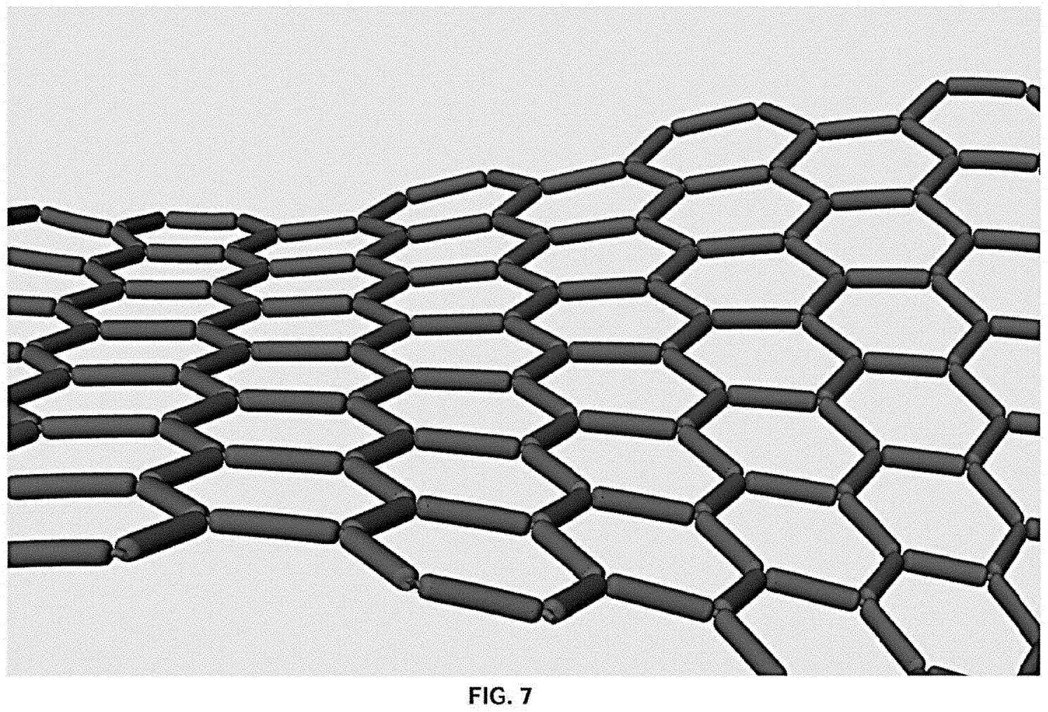

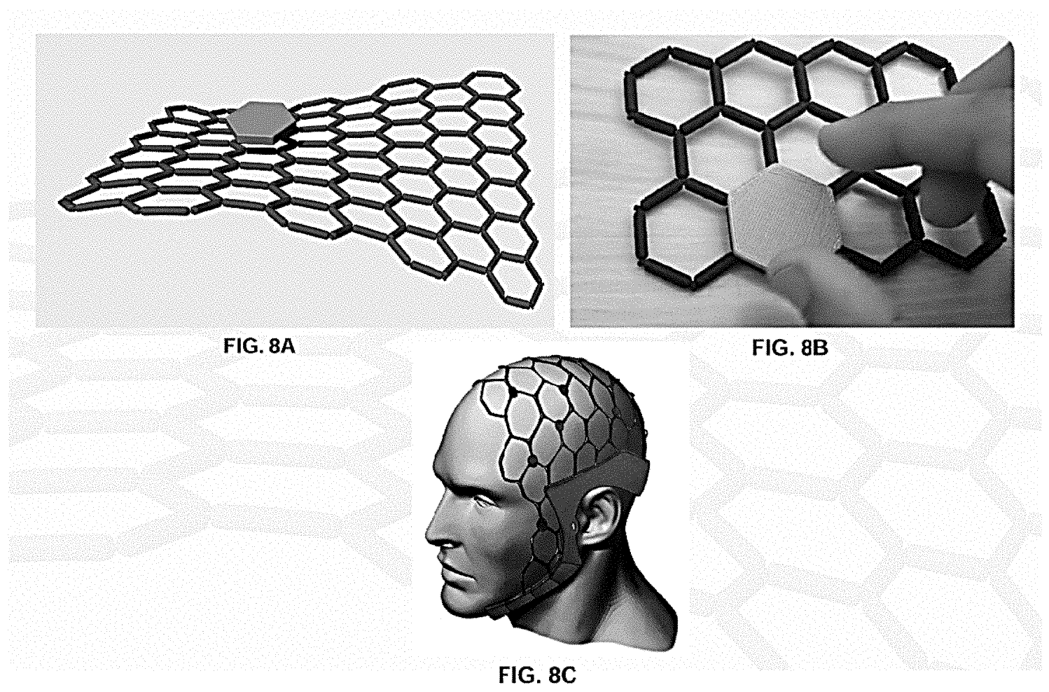

In another aspect, the invention relates to a hexagonal mesh system comprising one or more features, said one or more features having the ability to accommodate one or more devices or sensors, or to communicate with one or more devices or sensors. In one embodiment, the hexagonal mesh is a flexible mesh. In another embodiment, the hexagonal mesh is adoptable to one or more parts of a human or an animal body. As shown in FIG. 7, the hexagonal units can create one flexible form that can accommodate devices of diverse shapes, sizes, and placement on the body. As shown in FIG. 8A, the hexagon unit array allows contoured placement in close proximity to an organ of interest. As shown in FIG. 8B, hexagonal unit attachment points allow placement of different sensor devices. As shown in FIG. 8C, the hexagonal mesh is flexibly adoptable to the shapes and contours of parts of a human body.

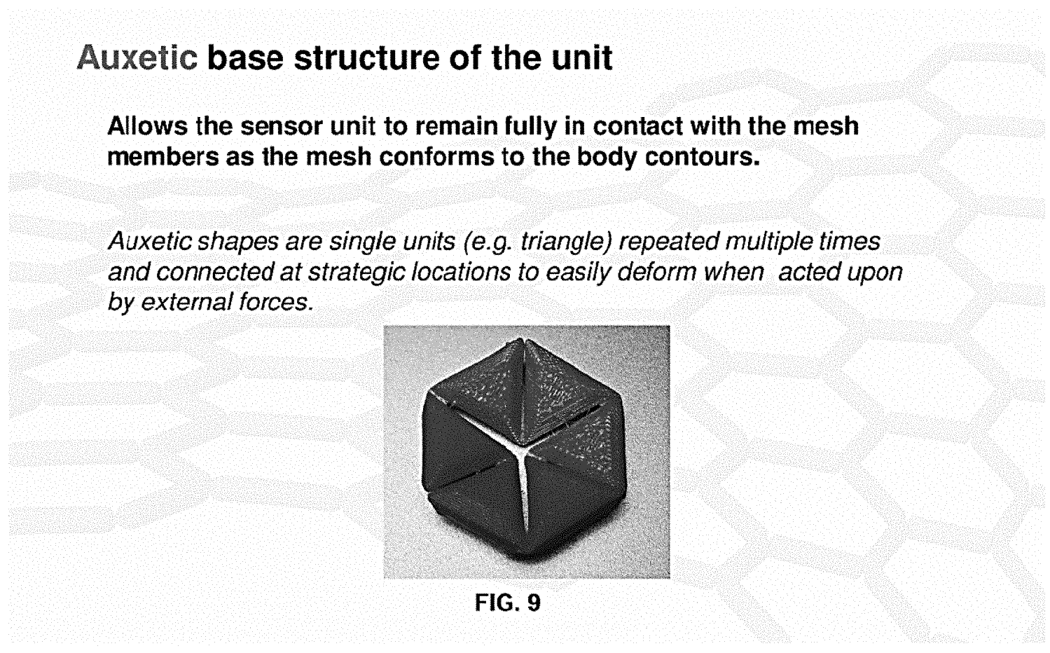

In another aspect, the invention provides an auxetic base structure. FIG. 9 shows an example of an auxetic base structure of the hexagonal unit. The structure may comprise a sensor unit. In some embodiments, the structure may allow the sensor unit to remain fully in contact with the mesh members as the mesh conforms to the body contours. In some embodiments, the auxetic shapes are single units (e.g. a triangle) repeated multiple times and connected at strategic locations to easily deform when acted upon by external forces.

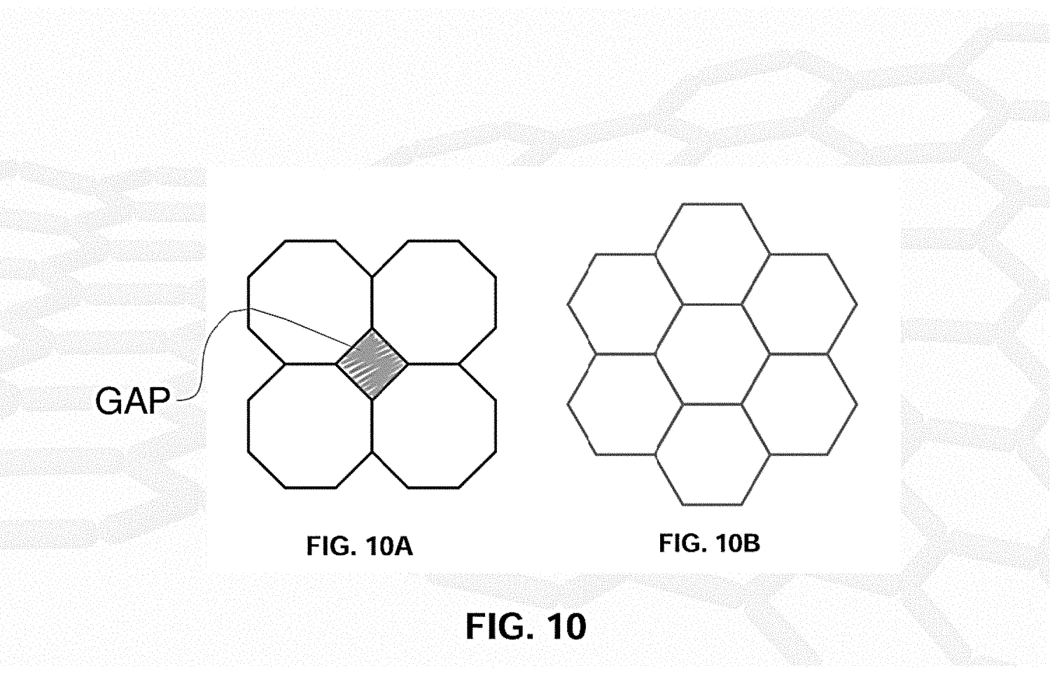

FIG. 10 illustrates an example of a hexagonal form. In some embodiments, as shown in FIG. 10B, hexagons fit together neatly without any gaps. In other embodiments, as shown in FIG. 10A, non-hexagon units have gaps, which create inconsistent shapes in the mesh.

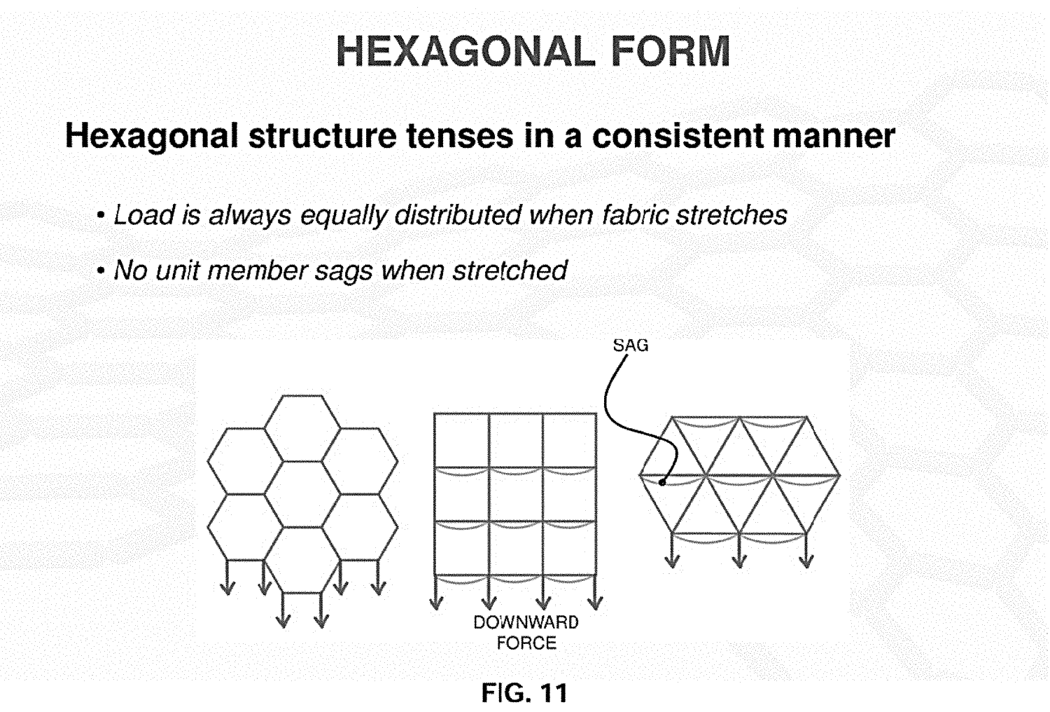

As shown in FIG. 11, the hexagonal structure tenses in a consistent manner. The hexagon structure load is always equally distributed when fabric stretches. However, anon-hexagon unit member sags when stretched.

It should be appreciated that embodiments formed from combinations of features set forth in separate embodiments are also within the scope of the present invention.

While certain features of the invention have been illustrated and described herein, modifications, substitutions, and equivalents are included within the scope of the invention.

1. An integrated health platform comprising:
a processor;
one or more hexagonal units that can create one flexible form (garment) that can accommodate a plurality of devices and sensors of diverse shapes, sizes, and placement, and permits close proximity of one or more sensors to the body, and allows electrical communication with the processor, each hexagonal unit formed from a plurality of conductive hexagonal cells;
two or more sensors, each of the sensors secured within one of the hexagonal cells, each sensor operative to detect a different health parameter; and
a visual output device, the output device configured to graphically display each of the health parameters in an intuitive format, and to permit access to more detailed data
one or more sensors embedded within a separate object that acts as a tangible avatar for interactive behavior and to provoke a response based on sensor data.

2. The platform of claim 1, wherein the processor is further operative: to identify one or more abnormal health parameters, each of the abnormal health parameters being non-compliant with an established health threshold, and to graphically designate the abnormal health parameters

3. The platform of claim 2, wherein the processor is further operative to graphically display the abnormal health parameters.

4. The platform of claim 2, wherein the processor is further operative to propose at least one therapeutic action to a user responsively to the abnormal health parameters.

5. The platform of claim 3, wherein the processor is further operative to graphically display a degree of non-compliance with the abnormal health parameters responsively to a user request.

6. The platform of claim 1, further comprising a plurality of therapeutic devices, each of the devices embedded in one of the hexagonal cells.

7. The platform of claim 1, wherein the processor is further operative to initiate a plurality of therapeutic devices, each of the devices embedded in one of the hexagonal cells.

8. The platform of claim 2, wherein the processor is further operative to actuate at least one of the therapeutic devices to administer a therapeutic treatment responsively to one or more abnormal health parameter.

9. The platform of claim 8, wherein the therapeutic treatment is selected from the group consisting of transference of energy such as by electricity, light, sound, infrared, electromagnetic pulse, or by bioactive or pharmaceutical administration.

10. The platform of claim 1, wherein the conductive grid is implemented as a conductive polymer.

11. A hexagonal mesh system comprising one or more features, said one or more features having ability to accommodate one or more devices or sensors, or to communicate with one or more devices or sensors, wherein said hexagonal mesh is flexibly adoptable to one or more parts or contours of a human or an animal body.
